(19) United States
(12) Patent Application Publication
(10) Pub. No.: US 2015/0282938 A1
(43) Pub. Date: Oct. 8, 2015

(54) EXPANDABLE SPACER

(71) Applicant: Atlas Spine, Inc., Jupiter, FL (US)

(72) Inventor: Matthew G. Baynham, Jupiter, FL (US)

(21) Appl. No.: 14/656,274

(22) Filed: Mar. 12, 2015

Related U.S. Application Data
(60) Provisional application No. 61/952,075, filed on Mar. 12, 2014.

(51) Int. Cl. A61F 2/44

(52) U.S. Cl. CPC A61F 2/442

(57) ABSTRACT

A spinal implant having a support body and a main body. The support body is positioned between an upper section and a lower section of the main body which are moveable in relation to the support body to increase the distance between the sections. The support body employs an aperture placed through the middle of the support body for receipt of a threaded link having inclined ramps for separating of the sections.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 61/952,075, entitled “Expandable Spacer”, filed Mar. 12, 2014. The contents of the above referenced application is incorporated herein by reference in its entirety.

### FIELD OF THE INVENTION

This invention relates to the field of orthopedic surgery and, more particularly, to implants to be placed between vertebrae in the spine.

### BACKGROUND OF THE INVENTION

Spinal stabilization is one approach to alleviating chronic back pain caused by displaced disk material or excessive movement of individual vertebrae. Conventional stabilization techniques include fusing two or more vertebrae together to circumvent or immobilize the area of excessive movement. Normally, the vertebral disk material which separates the vertebrae is removed and bone graft material is inserted in the space for interbody fusion. In addition to or, in place of, the bone graft material, a spinal implant may be inserted in the intervertebral space.

The conventional surgical approach for stabilization has been posteriorly for ease of access to the spine and to avoid interfering with internal organs and tissue. Usually the implant site is prepared to maintain natural lordosis and to accept a certain sized implant within certain pressure limits. This requires considerable time and skill by the surgeon.

U.S. Pat. No. 6,562,074 discloses a spinal insert which can be manipulated to adjust the height of the implant through links connected to the upper and lower plates.

U.S. Pat. No. 6,120,506 discloses a lordotic implant and a tap for use in preparing the vertebrae. The implant is designed to be inserted between the non-parallel end plates of adjacent vertebrae and maintain the natural lordotic angle of the spine. This is done through the use of a threaded tapered plug inserted in a tapped hole in the direction required by the lordosis of the spine. The implant is hollow and has radial apertures for accommodating bone graft material.

U.S. Pat. No. 6,015,436 discloses a tubular spinal implant. The implant is hollow and has radial apertures for interbody fusion through bone growth material. The device is placed between adjacent vertebrae with the opposite ends of the tube contacting the opposing vertebrae. The opposite ends are threaded together to form the hollow tube.

U.S. Pat. No. 7,850,733 discloses an opposing wedge ramp having a main body having upper and lower sections with mating sidewalls relatively movable along an inclined ramp. The inclined ramp forms a wedge movable between inclined sidewalls of the main body sections. The main body sections and the inclined ramp form a hollow cube-shaped structure with common open sides.

### SUMMARY OF THE INVENTION

Disclosed is an implant having a support body and a main body. The support body is positioned between an upper section and a lower section of the main body, said sections are moveable in relation to said support body to increase the distance between the sections. The support body includes an aperture placed through the middle of the support body for receipt of a threaded link, each end of the upper and lower section having an inclined ramp for receipt of a reciprocal ramp rotatably secured to each end of the threaded link.

The implant is inserted between adjacent vertebrae and the threaded link rotated until the desired distance between the sections is obtained. The threaded link is inserted between the sections through one end, the ramps permit the sections to move vertically away from each other for increasing the height of the implant and engaging the opposing surfaces of adjacent vertebrae. The adjacent vertebrae are forced apart as the height of the implant increases. The spinal fusion device may be used unilaterally or bilaterally.

Accordingly, it is an objective of the instant invention to teach a posterior surgical approach for placement of an adjustable spinal implant for interbody fusion allowing the implant to be inserted through a small incision and increased in size in situ.

It is a further objective of the instant invention to teach a spinal implant having increased contact area in the disk space.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a perspective view of the spinal implant in a compressed mode;

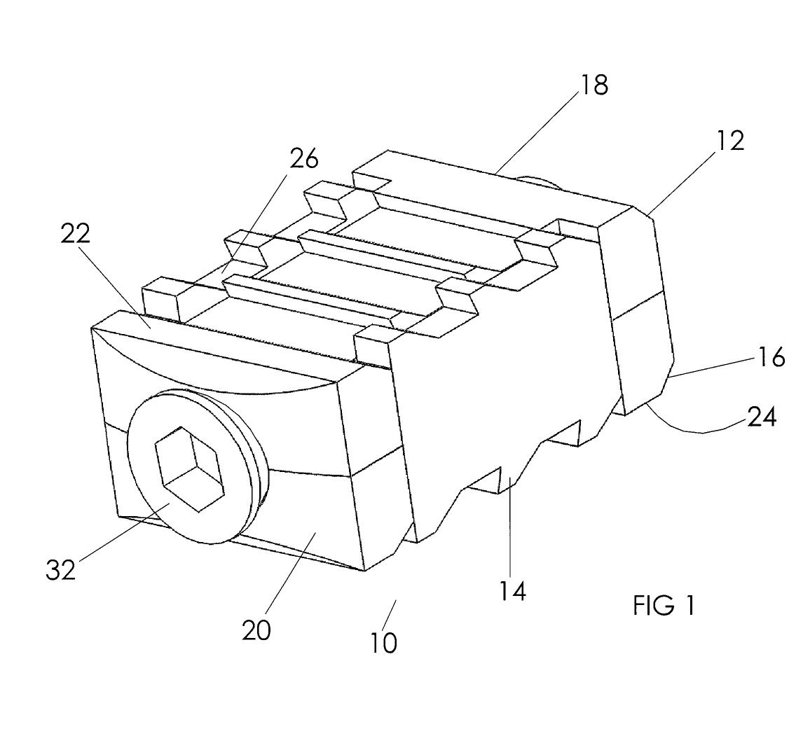

FIG. 2 is a skeleton view of the implant shown in FIG. 1;

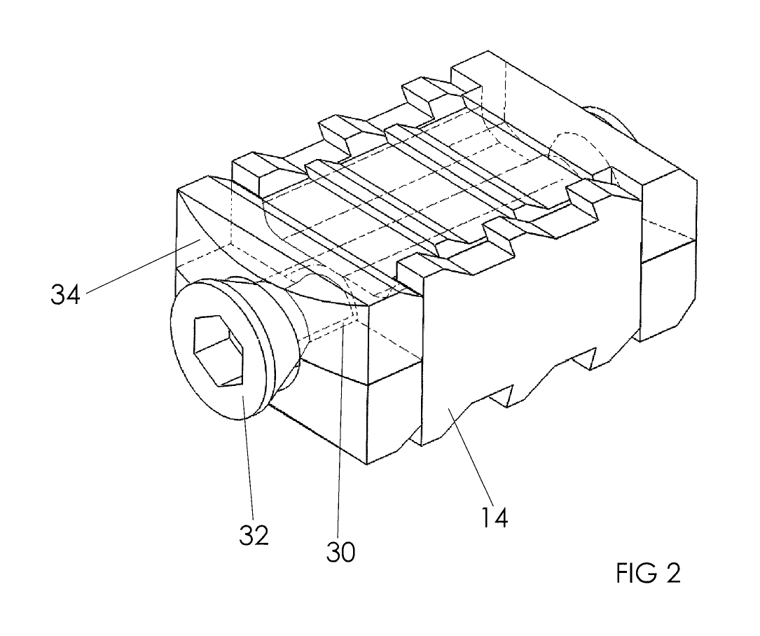

FIG. 3 is a perspective view of the spinal implant in an expanded mode;

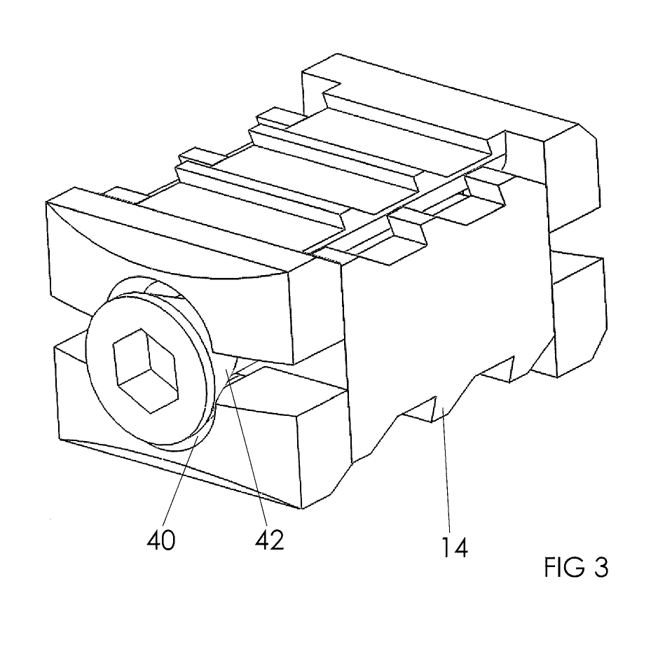

FIG. 4 is a side view of the spinal implant in an expanded mode;

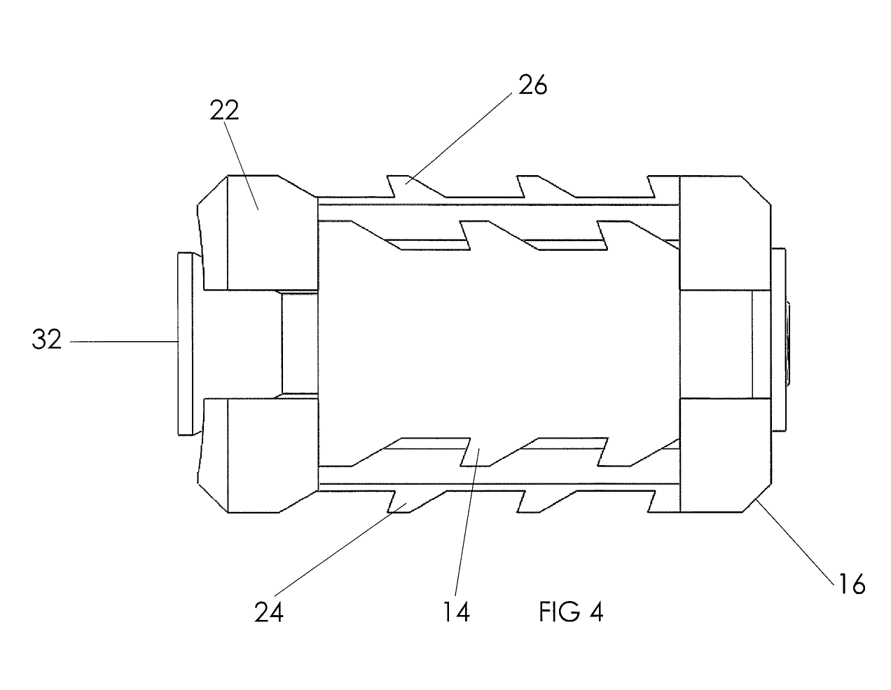

FIG. 5 is a skeleton view of the implant shown in FIG. 4; and

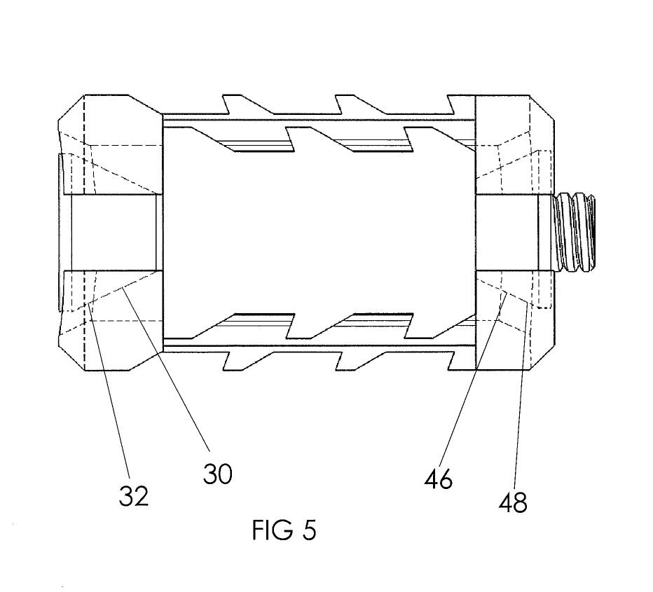

FIG. 6 is a perspective view of the spinal implant in an expanded mode.

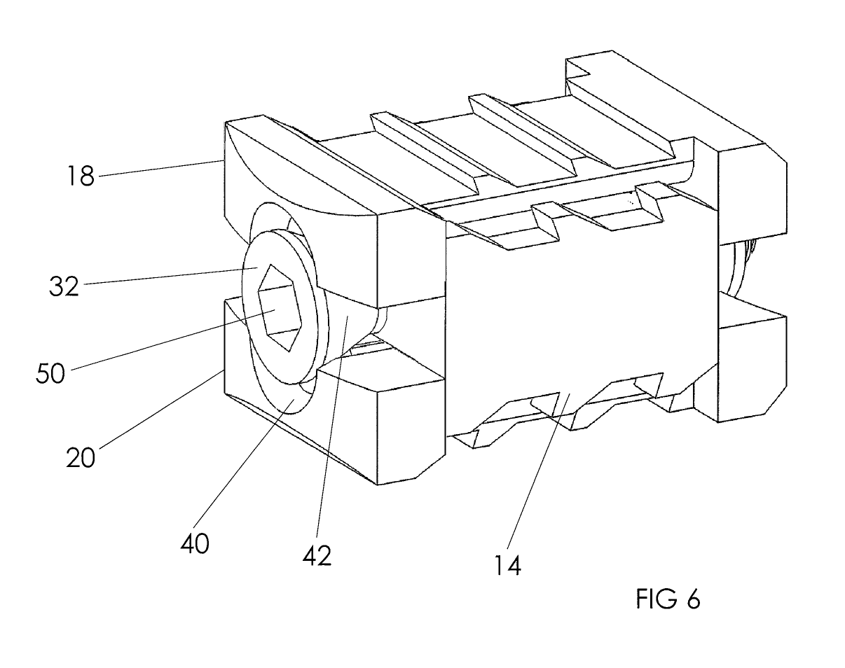

### DETAILED DESCRIPTION OF THE INVENTION

The spinal implant 10 is inserted in the intervertebral space to replace damaged, missing or excised disk material. This extended position allows the leading end 12 of the implant to be inserted in a small intervertebral space without the necessity of excising structurally sound bone. The implant 12 has a support body 14 and a main body 16. The support body 14 is positioned between an upper section 18 and a lower section 20 of the main body 16. The sections 18 and 20 are moveable in relation to the support body 14 to increase the distance between the sections 18 and 20.

The top surface 22 and the bottom surface 24 are planar to provide a large contact area with the vertebra. Each surface area has contact lands and grooves 26 to provide a better purchase although other stippled treatment may be employed.

The support body 14 includes an aperture 30 placed through the middle of the support body 14 for receipt of a threaded link 32, each end 34 and 36 of the upper 18 and lower section 20 having an inclined ramp 40 for receipt of a reciprocal ramp 42 rotatably secured to each end of the threaded link 32. Similarly the opposite end has an inclined ramp 46 for receipt of a reciprocal ramp 48. The device 10 has two extreme positions and is adjustable infinitely therebetween. The fusion device 10 may be made of conventional materials used for surgical implants, such as stainless steel and its many different alloys, titanium, and any other metal with the requisite strength and biologically inert properties. Polymeric materials with adequate strength and biological properties may also be used in the construction of the fusion device.

The implant 10 is inserted between adjacent vertebrae and the threaded link 32 is rotated by use of a driver receptacle 50 until the desired distance between the sections 18 and 20 is obtained. The threaded link 32 has ramps on each end to permit the sections to move vertically away from each other for increasing the height of the implant and engaging the opposing surfaces of adjacent vertebrae. The threaded link ramps are preferable conical shaped threaded nuts that allow for self centering of the ramps. The adjacent vertebrae are forced apart as the height of the implant increases. The spinal fusion device may be used unilaterally or bilaterally.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. The product comprising any feature described, either individually or in combination with any feature, in any configuration.

2. A procedure comprising any product described in any order, using any modality either individually or in any combination, with any modality or any feature described.
